(19) United States
(12) Patent Application Publication
(10) Pub. No.: US 2021/0372267 A1
(43) Pub. Date: Dec. 2, 2021

(54) SENSING DEVICE, SYSTEM AND METHOD FOR USE IN CIVIL ENGINEERING

(71) Applicant: Citpo Technologies Co., Ltd., Taipei City (TW)

(72) Inventors: Towny Huang, Taipei City (TW); Yen-Te Ho, Taipei City (TW)

(21) Appl. No.: 16/882,922

(22) Filed: May 26, 2020

(51) Int. Cl. E21B 47/01; E21B 47/06; E21B 23/04

(52) U.S. Cl. CPC E21B 47/01; G01N 33/24; E21B 23/0411; E21B 47/06

(57) ABSTRACT

The present invention is related to a sensing device, a system and a method for performing a measurement at a first predetermined depth in a monitoring well on a land for use in civil engineering, wherein the sensing device includes: a device main body; an inner sensor configured in the device main body; a first sleeve configured on the device main body and including a cylindrical hollow body having at least one axial through hole and a first and a second annular end surfaces; a first inflatable diaphragm configured on the first sleeve, wherein the monitoring well includes an inner wall, and when the first inflatable diaphragm is in a first inflation status, the first inflatable diaphragm presses against the inner wall at a second predetermined depth so as to define the land into an upper stratum thereabove and a lower stratum thereunder; and an outer sensor penetrating the at least one axial through hole for sensing an analyte flowing through the lower stratum.

### FIELD OF THE INVENTION

The present invention is related to a sensing device, a system and a method for use in civil engineering, particularly, to an environmental protection sensing device, system and method, with additional sensors configured in a sleeve or sleeves on a device main body.

### BACKGROUND OF THE INVENTION

In field of the civil engineering monitoring regarding groundwater pollution prevention and remediation in environmental engineering, debris flow prevention in earth engineering, leakage prevention and stabilization of reservoir dams or collapse prevention of retaining walls in hydraulic engineering, and so on, they are used in the field of civil engineering monitoring, if remediation of groundwater pollution in a large factory area is determined to be carried out, methods for applying chemicals (for example, in situ chemical oxidation (ISCO), or a surfactant flushing method (i.e. Surfactant-Enhanced Aquifer Remediation (SEAR)) and so on) can be used to the area. Thus, oxidants will be injected into the subsurface at multiple treatment points, so that the oxidants diffuse from each treatment point to the surrounding. To obtain the information regarding whether the remediation effect is good or not, or whether the oxidants are evenly applied, the following process is generally adopted. Two monitoring wells are dug under a ground surface area, and then a chemical or hot water is poured into one monitoring well with a higher water level. Thereafter, a pressure sensor or a temperature sensor is arranged in the other monitoring well with a lower water level for measuring the change of the pressure or temperature in the monitoring well.

The chemical or hot water injected into the monitoring well at the higher water level, however, does not flow along the horizontal direction in the stratum, but it is offset downward by gravity. In addition, to obtain the information regarding at which depth of the stratum the effect of the chemical presents, generally, sensing will be carried out by using sensing device, wherein a pressure sensor (for example, a pressure gauge) or a temperature sensor (for example, a thermometer) will be arranged in a main tube, and a packer is arranged on a upper outside of the main tube so that an isolated space is formed below the packer to prevent the water above the packer from leaking into this space. The packer is carried by using an inflatable membrane, wherein the membrane will be expanded after the inflation by using a pressuremeter, and the membrane is the same as that used in a pressuremeter test (PMT). However, after obtaining the pressure and temperature data, the pressure sensor or the temperature sensor must be removed from the main tube and a chemical sensor is then placed therein if it is desired to further measure the acidity of the stratum. It is clear that such an operation is inconvenient and inefficient.

In order to overcome the drawbacks in the prior art, the present invention provides a sensing device, a system and a method for use in civil engineering by which various types of sensors can act and be fixed simultaneously and conveniently.

### SUMMARY OF THE INVENTION

The present invention provides a sensing device for performing a measurement at a first predetermined depth in a monitoring well on a land for use in civil engineering, including: a device main body; an inner sensor configured in the device main body; a first sleeve configured on the device main body and including a cylindrical hollow body having at least one axial through hole and a first and a second annular end surfaces; a first inflatable diaphragm configured on the first sleeve, wherein the monitoring well includes an inner wall, and when the first inflatable diaphragm is in a first inflation status, the first inflatable diaphragm presses against the inner wall at a second predetermined depth so as to define the land into an upper stratum thereabove and a lower stratum thereunder; and an outer sensor penetrating the at least one axial through hole for sensing an analyte flowing through the lower stratum.

The present invention also provides a sensing system for use in civil engineering, including a plurality of sensing devices respectively arranged in a plurality of monitoring wells, wherein the plurality of monitoring wells are arranged around a reference point of a land in a predetermined manner, each monitoring well has an inner wall, and each sensing device performs a measurement at a predetermined depth in a respective monitoring well and includes a device main body; an inner sensor configured in the device main body; a first sleeve configured on the device main body and having at least one axial through hole, a cylindrical hollow body, and a first and a second annular end surfaces; a first inflatable diaphragm configured on the first sleeve, wherein when the first inflatable diaphragm is in a first inflation status, the first inflatable diaphragm presses against the respective inner wall to define the land into an upper stratum thereabove and a lower stratum thereunder; and an outer sensor penetrating the at least one axial through hole for sensing an analyte flowing through the lower stratum.

The present invention further provides a sensing method for use in civil engineering, including: selecting a land to be measured; determining a reference point on the land to be measured; arranging a plurality of monitoring wells around the reference point according to a predetermined arrangement; selecting a measurement point at a respective depth for each of the plurality of monitoring wells according to a predetermined plan; respectively placing a plurality of sensing devices of the present invention at the measurement points; and sensing an environmental parameter using the plurality of sensing devices.

### BRIEF DESCRIPTION OF THE DRAWINGS

The details and advantages of the present invention will become more readily apparent to one ordinarily skilled in the art after reviewing the following detailed descriptions and accompanying drawings.

FIG. 1 is a schematic diagram of a stratum under a plant area, wherein two monitoring wells are arranged.

FIG. 2A is a schematic front view of a sensing device for use in civil engineering according to a preferred embodiment of the present invention.

FIG. 2B is a schematic diagram of the operation mode of the sensing device of the present invention in the monitoring well in FIG. 1.

FIG. 3A is a schematic front view of a sensing device for use in civil engineering according to a preferred embodiment of the present invention.

FIG. 3B is a perspective schematic view of the sensing device according to a preferred embodiment of the present invention, wherein a first sleeve is provided, and axial through holes and an air outlet are configured in the first sleeve of the sensing device.

FIG. 3C a schematic diagram of the operation mode of the sensing device in FIG. 3A of the present invention in the monitoring well in FIG. 1.

FIG. 4 is a perspective schematic view of the sensing device according to a preferred embodiment of the present invention, wherein axial through holes and air outlets are configured in the sleeves of the sensing device.

FIG. 5 is a perspective schematic view of the sensing device according to a preferred embodiment of the present invention, wherein a plurality of air outlets are arranged on the air-injecting tube.

FIG. 6 is a perspective schematic view of a coupler and its quick connector that can be connected with the sensing device of the present invention.

FIG. 7 is a perspective schematic view of the quick coupler of the sensing device of the present invention.

FIG. 8 is a diagram showing an arrangement of a plurality of monitoring wells dug in a plant area for performing remediation of groundwater pollution for civil engineering.

FIG. 9 is a flowing chart of a sensing method for use in civil engineering of the present invention.

### DETAILED DESCRIPTION OF THE PREFERRED EMBODIMENT

In the conventional sensing processes for civil engineering, if various sensing processes, such as a pressure-sensing process, a temperature-sensing process and a chemical-sensing process, are to be carried out, it is necessary to change the sensors for different purposes to complete the desired sensing processes. That is, various sensing processes cannot be carried out at the same time. To solve this problem, the present invention provides a sensing device, a sensing system and a sensing method by which simultaneous operations of a pressure sensor, a temperature sensor, a chemical sensor and so on can be achieved.

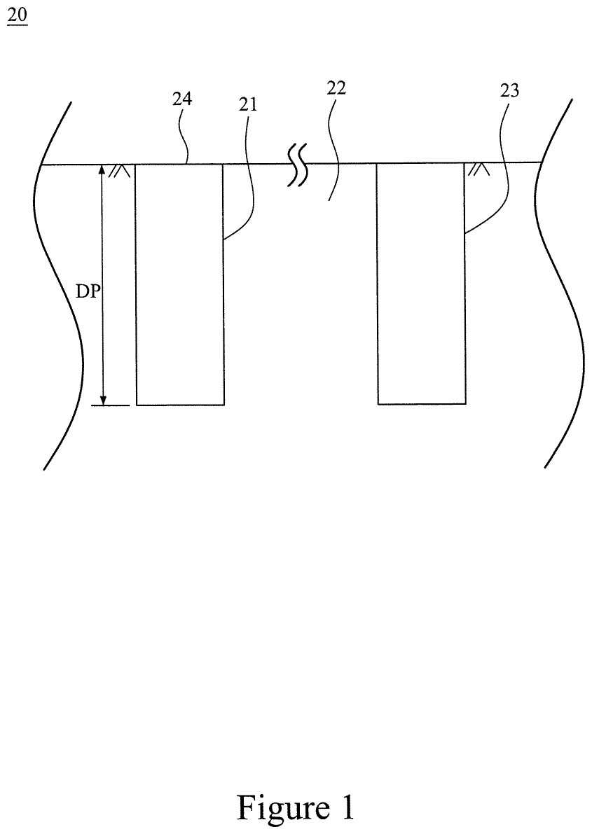

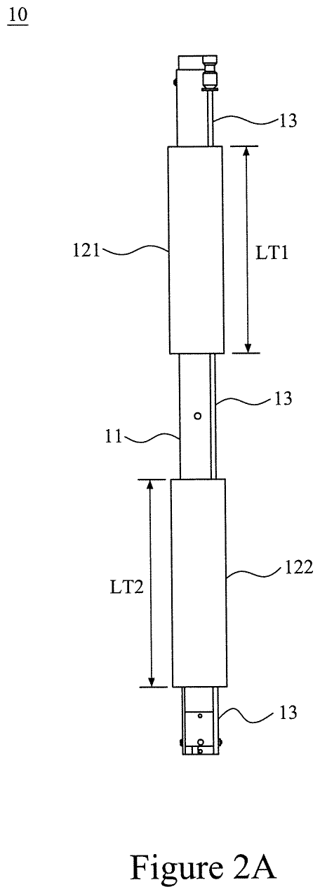

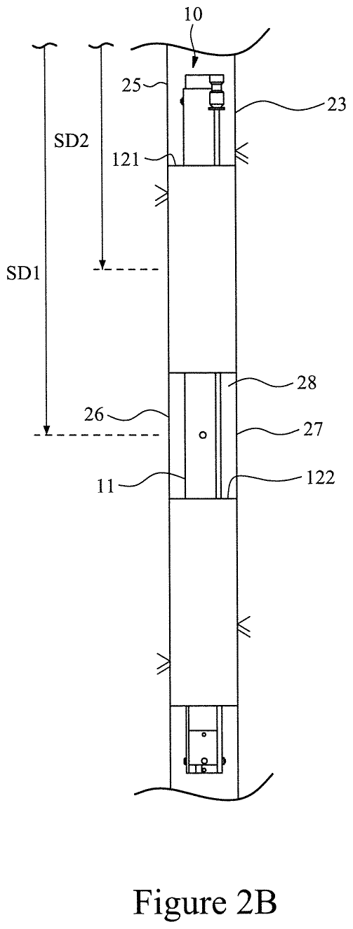

According to a preferred embodiment of the present invention, the structure and operations of the sensing device are described with reference to FIGS. 1, 2A and 2B. Generally, when the environmental remediation and monitoring in civil engineering are to be carried out in the stratum under a plant area or a hillside, monitoring wells would be arranged on the land of the plant area or the hillside first. FIG. 1 is a schematic diagram of a stratum under a plant area, wherein two monitoring wells are arranged for environmental remediation and monitoring in civil engineering. As shown in FIG. 1, there are two monitoring wells 21, 23 dug in the plant area 20. Each monitoring well 21, 23 in the plant area 20 has a round well opening 24 with a diameter of 50 mm and a depth DP of 15 meters, for example. When beginning the environmental remediation, chemical(s) or hot water is applied in the monitoring well 21. After the flowing of the applied chemical(s) or hot water in the stratum 22 for a time period, sensing is performed in the monitoring well 23 by using the sensing device 10 (as shown in FIG. 2A) for use in civil engineering according to the present invention. The sensing device 10 is placed in the monitoring well 23 (as shown in FIG. 2B) to perform the sensing at a predetermined depth SD1 (for example, 5 meters or 10 meters) under the land of the plant area 20. FIG. 2A is a schematic front view of the sensing device 10. Referring to FIG. 2A, the sensing device 10, which may be an environmental protection sensing device 10, includes a device main body 11, an inner sensor (not shown) configured in the device main body 11, a first sleeve (not shown) sleeved on the device main body 11, a second sleeve (not shown) sleeved on the device main body 11, a first inflatable diaphragm 121 wrapped on the first sleeve, a second inflatable diaphragm 122 wrapped on the second sleeve, an air-injecting tube 13 configured in the first sleeve and the second sleeve, and an outer sensor (not shown) configured in and penetrating the first sleeve. The device main body 11 is a main tube having a length, which may be any suitable length, for example 5 meters, for receiving the inner sensor, and an outer diameter, which may be any suitable diameter, for example, of 25 mm. The air-injecting tube 13 is used to inject air into the first inflatable diaphragm 121 and the second inflatable diaphragm 122 to facilitate the first inflatable diaphragm 121 and the second inflatable diaphragm 122 to be in an inflation status. More than one inner sensors may be configured in the device main body 11 at the same time. By referring to FIG. 2B, when the first inflatable diaphragm 121 and the second inflatable diaphragm 122 are in the inflation status, an isolated measurement space 28 among the device main body 11, an inner wall 27 of the monitoring well 23, the first sleeve, the second sleeve, the first inflatable diaphragm 121 and the second inflatable diaphragm 122 is formed.

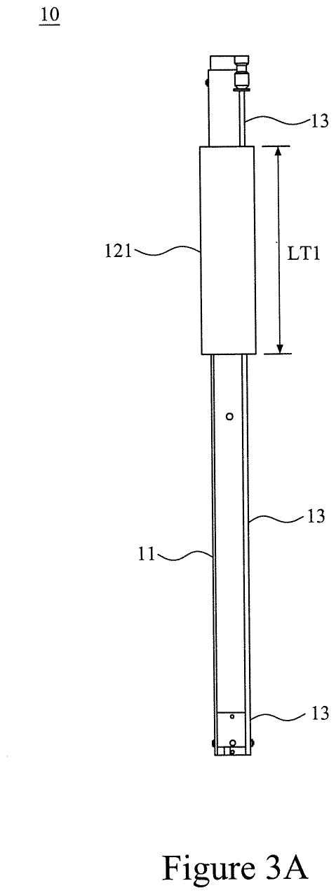

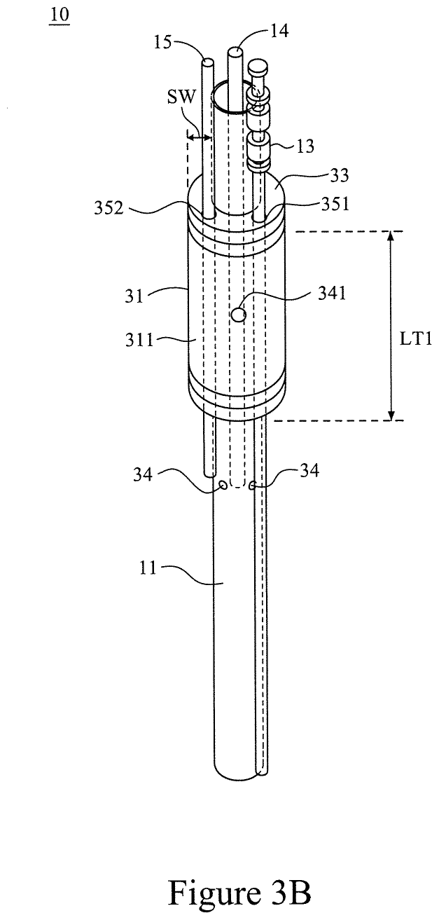

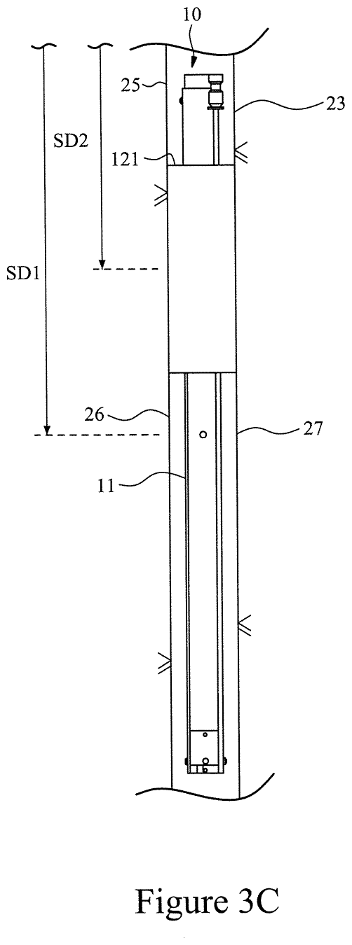

FIG. 3A is a schematic front view of a sensing device for use in civil engineering according to a preferred embodiment of the present invention; and FIG. 3B is a perspective schematic view of the sensing device according to a preferred embodiment of the present invention, wherein a first sleeve is provided, and axial through holes and an air outlet are configured in the first sleeve of the sensing device.

According to a preferred embodiment of the present invention, as shown in FIGS. 3A, 3B and 3C, the first sleeve 31 coaxially sleeved on the device main body 11 includes a cylindrical hollow body 311 for receiving the device main body 11; a first annular end surface 33 arranged on one end of the first sleeve 31 and a second annular end surface (not shown) arranged on the other end of the first sleeve 31, wherein the first annular end surface 33 and the second annular end surface are annular surfaces each having a width SW; at least one axial through hole, for example, two axial through holes 351, 352; and an air outlet 341 radially penetrating the cylindrical hollow body of the first sleeve 31. The axial through holes 351, 352 are configured in the first sleeve 31 within the width SW by axially penetrating the first annular end surface 33, the cylindrical hollow body 311 and the second annular end surface of the first sleeve 31. The inner sensor 14 is configured in the device main body 11 and put to the predetermined depth SD1. The outer sensor 15 is configured in the first sleeve 31 by penetrating the axial through hole 352 into a space below the first sleeve 31. The air-injecting tube 13 is configured in the first sleeve 31 by penetrating the axial through hole 351. If any further outer sensor(s) is needed to be used at the same time, additional axial through hole(s) can be provided in the first sleeve 31. A number of circulation holes 34 for the flowing of materials in the soil into the device main body 11 are configured around and radially penetrate the device main body 11 below the first sleeve 31. The number of circulation holes 34 may be any suitable number, for example, four circulation holes. By referring to FIGS. 3A, 3B and 3C, when the sensing device 10 is placed in the monitoring well 23 to perform the desired sensing, the first inflatable diaphragm 121 presses against the inner wall 27 of the monitoring well 23 at a predetermined depth SD2 so as to define the land into an upper stratum 25 (as shown in FIG. 3C) thereabove and a lower stratum 26 (as shown in FIG. 3C) thereunder when the first inflatable diaphragm 121 is in the inflation status. The predetermined depth SD1 is greater than the predetermined depth SD2. Thus, the inner sensor(s) 14 and the outer sensor(s) 15 may be used to sense the matter(s) flowing through the lower stratum 26 and thus environmental parameters are obtained.

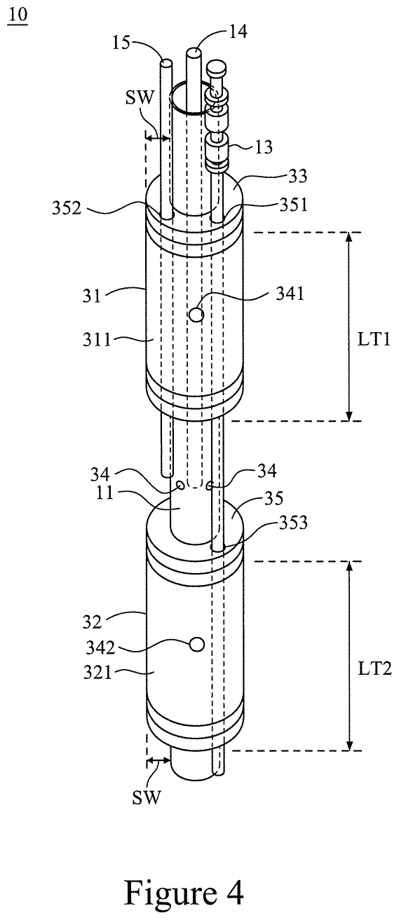

FIG. 4 shows the details of the first sleeve 31, the second sleeve 32 and the device main body 11 of the sensing device 10 in FIG. 2A according to a preferred embodiment of the present invention, wherein axial through holes 351, 352, circulation holes 34, and air outlets 341, 342 are configured, and the first inflatable diaphragm 121 and the second inflatable diaphragm 122 in FIG. 2A are omitted. According to a preferred embodiment of the present invention, as shown in FIG. 4, the first sleeve 31 and the second sleeve 32 are coaxially sleeved on the device main body 11 and separated from each other. For example, the first sleeve 31 and the second sleeve 32 may be coaxially sleeved on the upper part and lower part of the device main body 11, respectively, and separated from each other by a distance. The second sleeve 32 includes a cylindrical hollow body 321 for receiving the device main body 11. That is, the device main body 11 penetrates the hollow spaces of the first sleeve 31 and the second sleeve 32. The first sleeve 31 has a length, LT1, and the second sleeve 32 has a length, LT2. The length LT1 and the length LT2 may be any suitable lengths (for example, 150 mm), may be the same, or may be different from each other. The second sleeve 32 includes a first annular end surface 35 arranged on one end of the second sleeve 32 and a second annular end surface (not shown) arranged on the other end of the second sleeve 32, wherein the first annular end surface 35 and the second annular end surface of the second sleeve 32 are annular surfaces each having a width SW; at least one axial through hole, for example, an axial through hole 353, corresponding to the axial through hole 351 of the first sleeve 31; and an air outlet 342 radially penetrating the cylindrical hollow body 321 of the second sleeve 32. The axial through hole 353 is configured in the second sleeve 32 within the width SW by axially penetrating the first annular end surface 35, the cylindrical hollow body 321 and the second annular end surface of the second sleeve 32. The air-injecting tube 13 is configured in the first sleeve 31 and the second sleeve 32 by penetrating the axial through hole 351 of the first sleeve 31 and the axial through hole 353 of the second sleeve 32. If any further outer sensor(s) is needed to be used at the same time, additional axial through hole(s) can be correspondingly provided in the first sleeve 31 and the second sleeve 32. The air-injecting tube 13 is configured in the first sleeve 31 and the second sleeve 32 and is used to inject air into the first inflatable diaphragm 121 and the second inflatable diaphragm 122 to facilitate the first inflatable diaphragm 121 and the second inflatable diaphragm 122 to be in the inflation status. By referring to FIGS. 2B and 4, when the sensing device 10 is placed in the monitoring well 23 to perform the desired sensing, the first inflatable diaphragm 121 and the second inflatable diaphragm 122 press against the inner wall 27 of the monitoring well 23 under the inflation status, and thus an isolated measurement space 28 among the device main body 11, the inner wall 27, the first inflatable diaphragm 121, the second inflatable diaphragm 122, the first sleeve 31 and the second sleeve 32 is formed. The inner sensor 14 is configured in the device main body 11 and the outer sensor 15 penetrates the axial through hole 352 of the first sleeve 31 into isolated measurement space 28 to sense the analyte(s) therein, and thus environmental parameters are obtained.

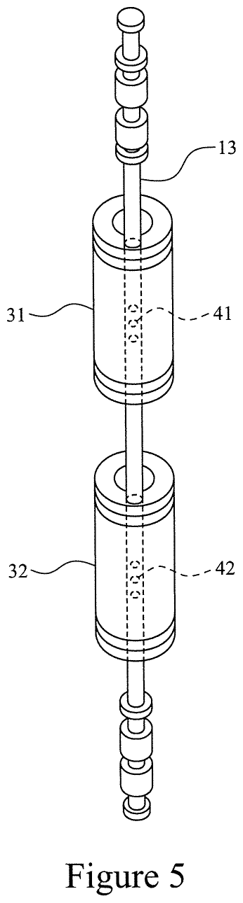

FIG. 5 shows the air-injecting tube 13 in which a plurality of air outlets 41, 42 are configured, wherein the air outlet 41 is arranged within the part of the air-injecting tube 13 corresponding to the first sleeve 31, and the air outlet 42 is arranged within the part of the air-injecting tube 13 corresponding to the second sleeve 32. By referring to FIGS. 1, 4 and 5, the air-injecting tube 13 injects air injects air through the air outlet 41 of the air-injecting tube 13 and the air outlet 341 of the first sleeve 31 into the first inflatable diaphragm 121 to facilitate the first inflatable diaphragm 121 to be in the inflation status, and through the air outlet 42 of the air-injecting tube 13 and the air outlet 342 of the second sleeve 32 into the second inflatable diaphragm 122 to facilitate the second inflatable diaphragm 122 to be in the inflation status.

The inner sensor may be at least one of a pressure sensor, a temperature sensor, a strain gauge and a displacement meter depending on the type(s) of measurement(s) to be performed. The outer sensor, which is at least one of a chemical sensor and a corrosion rate meter, and the inner sensor, which is at least one of a pressure sensor, a temperature sensor, a strain gauge and a displacement meter, may be conventional electronic sensors, or may be sensors each equipped with a Bragg Fiber Grating (FBG). The FBG may have a length of 20 mm, for example. The chemical sensor and the corrosion rate measuring meter are used to measure the acidity and corrosion rate of the underground, respectively. The analyte is a chemical substance or a hot water. The inner sensor is used to measure a change of a pressure or a temperature resulting from the chemical substance or the hot water.

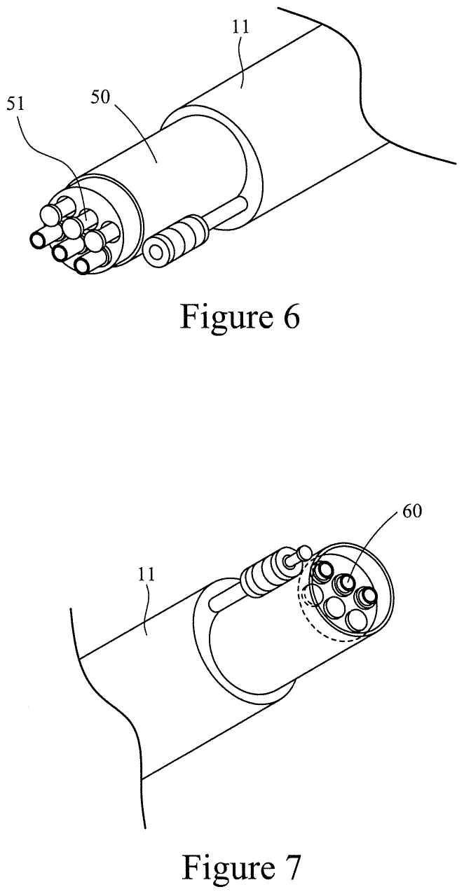

Referring to FIG. 6, the sensing device 10 further includes a coupler 50, which is connected to the device main body 11, for enabling the environmental protection sensing device 10 to work at the predetermined depth SD1 in the monitoring well 23. The coupler 50 has a length, which may be any suitable length, for example, depending on the depth at which the sensing device 10 is to work. For example, the length of the coupler 50 may be the same as that of the device main body 11, or be at least 3 m as needed. The coupler 50 has a quick connector 51. Referring to FIG. 7, the device main body 11 has a quick connector 60 for the connection with the quick connector 51 of the coupler 50.

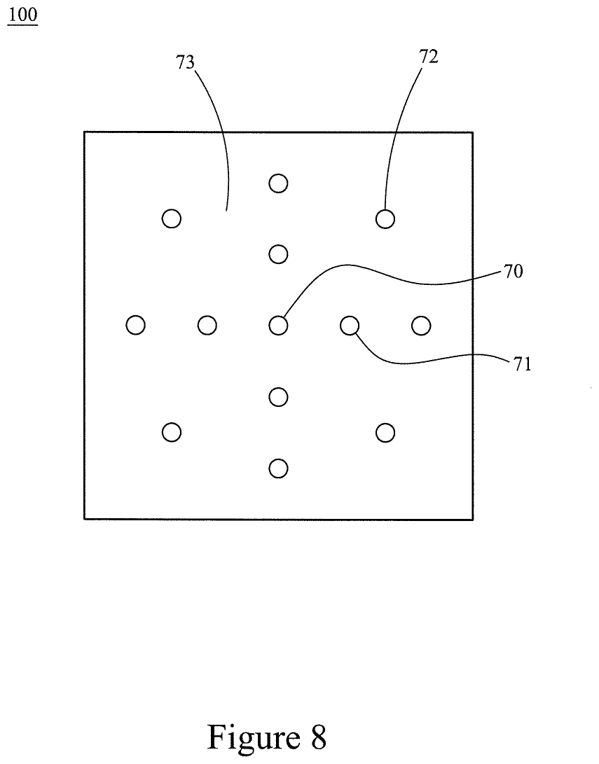

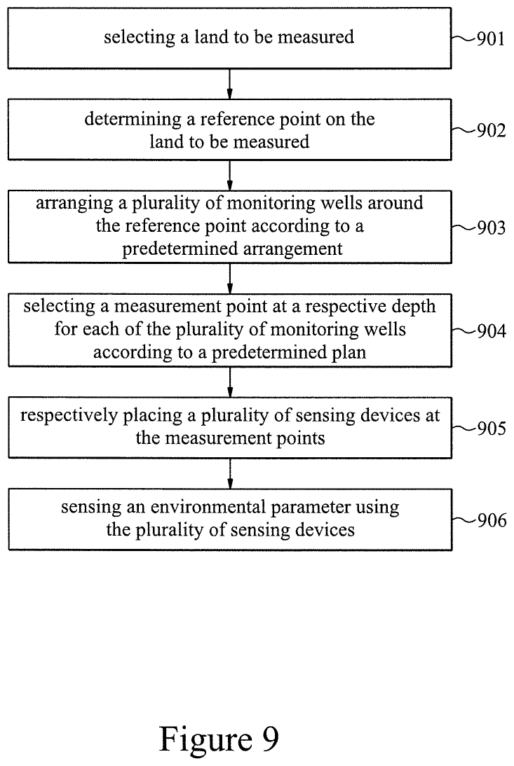

In one embodiment, the present invention provides a sensing system and method for environmental protection, which can be applied to the sensing of groundwater pollution remediation, debris flow monitoring and so on. FIGS. 8 and 9 respectively are a diagram and a flowing chart showing an arrangement of a plurality of monitoring wells dug in a plant area for performing remediation of groundwater pollution in civil engineering by using the sensing system and method of the present invention. The sensing system 100 of the present invention includes a plurality of sensing devices 10 as described with reference to FIGS. 2A-7 above. As shown in FIGS. 8 and 9, when a land 73 (i.e., a land in a selected plant area, or a land on which environmental protection sensing is to be carried out) is selected to be measured (Step 901), a reference point (an agent-applying well 70 as shown in FIG. 8) on the land 73 is determined (Step 902), and a plurality of monitoring wells (such as monitoring wells 71 and 72 as shown in FIG. 8) are arranged around the reference point on the land 73 in a predetermined manner (Step 903), wherein an agent, such as a chemical, a hot water and so on, for the groundwater pollution remediation, for example, is applied to the agent-applying well 70, and the agent flowing from the agent-applying well 70 is sensed in the monitoring wells 71 and 72. For example, the monitoring well 71 is located away from the agent-applying well 70 by a distance of 5 m, and the monitoring well 72 is located away from the agent-applying well 70 by a distance of 10 m. According to a predetermined plan, when a measurement point is selected at a respective depth for each of the plurality of monitoring wells 71 and 72 (Step 904), the plurality of sensing devices 10 of the sensing system 100 are placed into the monitoring wells 70, 71 at the measurement points (Step 905). In an embodiment, each sensing device 10 performs a measurement at a predetermined depth SD1 in the respective monitoring well 71, 72 in which the sensing device 10 is arranged. Alternatively, a plurality of measurement points located at different depths under the land 73 (for example, 5 measurement points at different depths) are selected for the monitoring wells 71, 72 according to a predetermined plan. After the selection of the measurement points, the sensing devices 10 are arranged in the monitoring wells 71, 72 at the selected measurement points to perform the sensing of environmental parameters at the same time (Step 906).

Based on the above, the present invention provides the effects that various monitoring processes for civil engineering can be conveniently, efficiently and simultaneously carried out by the inventive sensing device, system and method according to the present invention. Through the configuration of axial through holes in the sleeves of the device main body, various sensors for performing various sensing processes can be functioning at the same time. In addition, the use the axial through holes in the sleeves of the device main body has the effect of fastening the outer sensor(s) and the air-injecting tube.

### EMBODIMENTS

1. A sensing device for performing a measurement at a first predetermined depth in a monitoring well on a land for use in civil engineering, including: a device main body; an inner sensor configured in the device main body; a first sleeve configured on the device main body and including a cylindrical hollow body having at least one axial through hole and a first and a second annular end surfaces; a first inflatable diaphragm configured on the first sleeve, wherein the monitoring well includes an inner wall, and when the first inflatable diaphragm is in a first inflation status, the first inflatable diaphragm presses against the inner wall at a second predetermined depth so as to define the land into an upper stratum thereabove and a lower stratum thereunder; and an outer sensor penetrating the at least one axial through hole for sensing an analyte flowing through the lower stratum.
2. The sensing device according to Embodiment 1, further including an air-injecting tube penetrating the first sleeve.
3. The sensing device according to Embodiment 1 or 2, wherein the at least one axial through hole includes a first axial through hole and a second axial through hole, wherein the outer sensor penetrates the first axial through hole and the air-injecting tube penetrates the second axial through hole, the air-injecting tube has a first air outlet, the first sleeve further includes a second air outlet, and the air-injecting tube injects an air through the first air outlet and the second air outlet into the first inflatable diaphragm to facilitate the first inflatable diaphragm to be in the first inflation status, under which the first inflatable diaphragm presses against the inner wall of the monitoring well.
4. The sensing device according to any one of Embodiments 1-3, further including a second sleeve configured on the device main body and spaced apart from the first sleeve, a second inflatable diaphragm configured on the second sleeve, and an air-injecting tube, wherein the second sleeve has a cylindrical hollow body having at least one axial through hole and a first and a second annular end surfaces, and the air-injecting tube penetrates the first sleeve and the second sleeve.
5. The sensing device according to any one of Embodiments 1-4, wherein each of the at least one axial through holes of the first and the second sleeves includes a first axial through hole and a second axial through hole, the outer sensor penetrates the first axial through holes of the first and the second sleeves, and the air-injecting tube penetrates the second axial through holes of the first and the second sleeves, the air-injecting tube has a first air outlet and a second air outlet, the first sleeve further includes a third air outlet, the second sleeve further includes a fourth air outlet, and the air-injecting tube injects an air through the first air outlet and the third air outlet into the first inflatable diaphragm to facilitate the first inflatable diaphragm to be in the first inflation status, and through the second air outlet and the fourth air outlet into the second inflatable diaphragm to facilitate the second inflatable diaphragm to be in a second inflation status, under which the second inflatable diaphragm presses against the inner wall of the monitoring well.
6. The sensing device according to any one of Embodiments 1-5, wherein the first inflatable diaphragm and the second inflatable diaphragm press against the inner wall of the monitoring well under the first and the second inflation statuses respectively, an isolated measurement space among the device main body, the inner wall, the first inflatable diaphragm, the second inflatable diaphragm, the first sleeve and the second sleeve is formed, and the outer sensor penetrates into the isolated measurement space.
7. The sensing device according to any one of Embodiments 1-6, wherein the inner sensor is at least one of a pressure sensor, a temperature sensor, a strain gauge and a displacement meter, and each inner sensor is equipped with a Bragg Fiber Grating (FBG).
8. The sensing device according to any one of Embodiments 1-7, wherein the outer sensor is at least one of a chemical sensor and a corrosion rate meter, and each outer sensor is equipped with a Bragg Fiber Grating (FBG), for measuring an underground acidity and a corrosion rate.
9. The sensing device according to any one of Embodiments 1-8, wherein the analyte is a chemical substance or a hot water, and the inner sensor is used to measure a change of a pressure or a temperature resulting from the chemical substance or the hot water.
10. The sensing device according to any one of Embodiments 1-9, further including a coupler coupled to the device main body for enabling the sensing device to work at the first predetermined depth in the monitoring well, wherein the coupler has a quick connector to facilitate a connection between the device main body and the coupler.
11. A sensing system for use in civil engineering, including a plurality of sensing devices respectively arranged in a plurality of monitoring wells, wherein the plurality of monitoring wells are arranged around a reference point of a land in a predetermined manner, each monitoring well has an inner wall, and each sensing device performs a measurement at a predetermined depth in a respective monitoring well and includes: a device main body; an inner sensor configured in the device main body; a first sleeve configured on the device main body and having at least one axial through hole, a cylindrical hollow body, and a first and a second annular end surfaces; a first inflatable diaphragm configured on the first sleeve, wherein when the first inflatable diaphragm is in a first inflation status, the first inflatable diaphragm presses against the respective inner wall to define the land into an upper stratum thereabove and a lower stratum thereunder; and an outer sensor penetrating the at least one axial through hole for sensing an analyte flowing through the lower stratum.
12. The sensing system according to Embodiment 11, wherein each sensing device further includes an air-injecting tube penetrating the first sleeve.
13. The sensing system according to Embodiment 11 or 12, wherein the at least one axial through hole includes a first axial through hole and a second axial through hole, the outer sensor penetrates the first axial through hole and the air-injecting tube penetrates the second axial through hole, the air-injecting tube has a first air outlet, the first sleeve further includes a second air outlet, and the air-injecting tube injects an air through the first air outlet and the second air outlet into the first inflatable diaphragm to facilitate the first inflatable diaphragm to be in the first inflation status, under which the first inflatable diaphragm presses against the respective inner wall of the respective monitoring well.
14. The sensing system according to any one of Embodiments 11-13, wherein each sensing device further includes a second sleeve configured on the device main body and spaced apart from the first sleeve, a second inflatable diaphragm configured on the second sleeve, and an air-injecting tube, wherein the second sleeve has a cylindrical hollow body having at least one axial through hole and a first and a second annular end surfaces, and the air-injecting tube penetrates the first sleeve and the second sleeve.
15. The sensing system according to any one of Embodiments 11-14, wherein each of the at least one axial through holes of the first and second sleeves includes a first axial through hole and a second axial through hole, the outer sensor penetrates the first axial through holes of the first and the second sleeves, the air-injecting tube penetrates the second axial through holes of the first and second sleeves, the air-injecting tube has a first air outlet and a second air outlet, the first sleeve further includes a third air outlet, the second sleeve further includes a fourth air outlet, and the air-injecting tube injects an air through the first air outlet and the third air outlet into the first inflatable diaphragm to facilitate the first inflatable diaphragm to be in the first inflation status, and through the second air outlet and the fourth air outlet into the second inflatable diaphragm to facilitate the second inflatable diaphragm to be in a second inflation status, under which the second inflatable diaphragm presses against the respective inner wall of the respective monitoring well.
16. The sensing system according to any one of Embodiments 11-15, wherein the first inflatable diaphragm and the second inflatable diaphragm press against the respective inner walls of the respective monitoring wells under the first and the second inflation statuses respectively, a respective isolated measurement space among the respective device main body, the respective inner wall, the respective first inflatable diaphragm, the respective second inflatable diaphragm, the respective first sleeve and the respective second sleeve is formed, and the respective outer sensor penetrates into the respective isolated measurement space.
17. The sensing system according to any one of Embodiments 11-16, wherein each inner sensor is at least one of a pressure sensor, a temperature sensor, a strain gauge and a displacement meter, and is equipped with a Bragg Fiber Grating (FBG); and each outer sensor is at least one of a chemical sensor and a corrosion rate meter, is equipped with a Bragg Fiber Grating (FBG) and is used for measuring an underground acidity and a corrosion rate.
18. The sensing system according to any one of Embodiments 11-17, wherein the analyte is a chemical substance or a hot water, and each inner sensor is used to measure a change of a pressure or a temperature resulting from the chemical substance or the hot water.
19. The sensing system according to any one of Embodiments 11-18, wherein each sensing device further includes a coupler coupled to the device main body, for enabling the sensing device to work at the predetermined depth in the respective monitoring well, and the coupler has a quick connector to facilitate a connection between the device main body and the coupler.
20. A sensing method for use in civil engineering, including: selecting a land to be measured; determining a reference point on the land to be measured; arranging a plurality of monitoring wells around the reference point according to a predetermined arrangement; selecting a measurement point at a respective depth for each of the plurality of monitoring wells according to a predetermined plan; respectively placing a plurality of sensing devices according to any one of Embodiments 1-10 at the measurement points; and sensing an environmental parameter using the plurality of sensing devices.

It is contemplated that modifications and combinations will readily occur to one ordinarily skilled in the art, and these modifications and combinations are within the scope of this invention.

What is claimed is:

1. A sensing device for performing a measurement at a first predetermined depth in a monitoring well on a land for use in civil engineering, comprising:
a device main body;
an inner sensor configured in the device main body;
a first sleeve configured on the device main body and including a cylindrical hollow body having at least one axial through hole and a first and a second annular end surfaces;
a first inflatable diaphragm configured on the first sleeve, wherein the monitoring well includes an inner wall, and when the first inflatable diaphragm is in a first inflation status, the first inflatable diaphragm presses against the inner wall at a second predetermined depth so as to define the land into an upper stratum thereabove and a lower stratum thereunder; and
an outer sensor penetrating the at least one axial through hole for sensing an analyte flowing through the lower stratum.

2. The sensing device according to claim 1, further comprising an air-injecting tube penetrating the first sleeve.

3. The sensing device according to claim 2, wherein the at least one axial through hole includes a first axial through hole and a second axial through hole, wherein the outer sensor penetrates the first axial through hole and the air-injecting tube penetrates the second axial through hole, the air-injecting tube has a first air outlet, the first sleeve further includes a second air outlet, and the air-injecting tube injects an air through the first air outlet and the second air outlet into the first inflatable diaphragm to facilitate the first inflatable diaphragm to be in the first inflation status, under which the first inflatable diaphragm presses against the inner wall of the monitoring well.

4. The sensing device according to claim 1, further comprising a second sleeve configured on the device main body and spaced apart from the first sleeve, a second inflatable diaphragm configured on the second sleeve, and an air-injecting tube, wherein the second sleeve has a cylindrical hollow body having at least one axial through hole and a first and a second annular end surfaces, and the air-injecting tube penetrates the first sleeve and the second sleeve.

5. The sensing device according to claim 4, wherein each of the at least one axial through holes of the first and the second sleeves includes a first axial through hole and a second axial through hole, the outer sensor penetrates the first axial through holes of the first and the second sleeves, and the air-injecting tube penetrates the second axial through holes of the first and the second sleeves, the air-injecting tube has a first air outlet and a second air outlet, the first sleeve further includes a third air outlet, the second sleeve further includes a fourth air outlet, and the air-injecting tube injects an air through the first air outlet and the third air outlet into the first inflatable diaphragm to facilitate the first inflatable diaphragm to be in the first inflation status, and through the second air outlet and the fourth air outlet into the second inflatable diaphragm to facilitate the second inflatable diaphragm to be in a second inflation status, under which the second inflatable diaphragm presses against the inner wall of the monitoring well.

6. The sensing device according to claim 5, wherein the first inflatable diaphragm and the second inflatable diaphragm press against the inner wall of the monitoring well under the first and the second inflation statuses respectively, an isolated measurement space among the device main body, the inner wall, the first inflatable diaphragm, the second inflatable diaphragm, the first sleeve and the second sleeve is formed, and the outer sensor penetrates into the isolated measurement space.

7. The sensing device according to claim 1, wherein the inner sensor is at least one of a pressure sensor, a temperature sensor, a strain gauge and a displacement meter, and each inner sensor is equipped with a Bragg Fiber Grating (FBG).

8. The sensing device according to claim 1, wherein the outer sensor is at least one of a chemical sensor and a corrosion rate meter, and each outer sensor is equipped with a Bragg Fiber Grating (FBG), for measuring an underground acidity and a corrosion rate.

9. The sensing device according to claim 1, wherein the analyte is a chemical substance or a hot water, and the inner sensor is used to measure a change of a pressure or a temperature resulting from the chemical substance or the hot water.

10. The sensing device according to claim 1, further comprising a coupler coupled to the device main body for enabling the sensing device to work at the first predetermined depth in the monitoring well, wherein the coupler has a quick connector to facilitate a connection between the device main body and the coupler.

11. A sensing system for use in civil engineering, comprising a plurality of sensing devices respectively arranged in a plurality of monitoring wells, wherein the plurality of monitoring wells are arranged around a reference point of a land in a predetermined manner, each monitoring well has an inner wall, and each sensing device performs a measurement at a predetermined depth in a respective monitoring well, and comprises:
a device main body;
an inner sensor configured in the device main body;
a first sleeve configured on the device main body and having at least one axial through hole, a cylindrical hollow body, and a first and a second annular end surfaces;
a first inflatable diaphragm configured on the first sleeve, wherein when the first inflatable diaphragm is in a first inflation status, the first inflatable diaphragm presses against the respective inner wall to define the land into an upper stratum thereabove and a lower stratum thereunder; and
an outer sensor penetrating the at least one axial through hole for sensing an analyte flowing through the lower stratum.

12. The sensing system according to claim 11, wherein each sensing device further comprises an air-injecting tube penetrating the first sleeve.

13. The sensing system according to claim 12, wherein the at least one axial through hole includes a first axial through hole and a second axial through hole, the outer sensor penetrates the first axial through hole and the air-injecting tube penetrates the second axial through hole, the air-injecting tube has a first air outlet, the first sleeve further includes a second air outlet, and the air-injecting tube injects an air through the first air outlet and the second air outlet into the first inflatable diaphragm to facilitate the first inflatable diaphragm to be in the first inflation status, under which the first inflatable diaphragm presses against the respective inner wall of the respective monitoring well.

14. The sensing system according to claim 11, wherein each sensing device further comprises a second sleeve configured on the device main body and spaced apart from the first sleeve, a second inflatable diaphragm configured on the second sleeve, and an air-injecting tube, wherein the second sleeve has a cylindrical hollow body having at least one axial through hole and a first and a second annular end surfaces, and the air-injecting tube penetrates the first sleeve and the second sleeve.

15. The sensing system according to claim 14, wherein each of the at least one axial through holes of the first and second sleeves includes a first axial through hole and a second axial through hole, the outer sensor penetrates the first axial through holes of the first and the second sleeves, the air-injecting tube penetrates the second axial through holes of the first and second sleeves, the air-injecting tube has a first air outlet and a second air outlet, the first sleeve further includes a third air outlet, the second sleeve further includes a fourth air outlet, and the air-injecting tube injects an air through the first air outlet and the third air outlet into the first inflatable diaphragm to facilitate the first inflatable diaphragm to be in the first inflation status, and through the second air outlet and the fourth air outlet into the second inflatable diaphragm to facilitate the second inflatable diaphragm to be in a second inflation status, under which the second inflatable diaphragm presses against the respective inner wall of the respective monitoring well.

16. The sensing system according to claim 15, wherein the first inflatable diaphragm and the second inflatable diaphragm press against the respective inner walls of the respective monitoring wells under the first and the second inflation statuses respectively, a respective isolated measurement space among the respective device main body, the respective inner wall, the respective first inflatable diaphragm, the respective second inflatable diaphragm, the respective first sleeve and the respective second sleeve is formed, and the respective outer sensor penetrates into the respective isolated measurement space.

17. The sensing system according to claim 11, wherein each inner sensor is at least one of a pressure sensor, a temperature sensor, a strain gauge and a displacement meter, and is equipped with a Bragg Fiber Grating (FBG); and each outer sensor is at least one of a chemical sensor and a corrosion rate meter, is equipped with a Bragg Fiber Grating (FBG) and is used for measuring an underground acidity and a corrosion rate.

18. The sensing system according to claim 11, wherein the analyte is a chemical substance or a hot water, and each inner sensor is used to measure a change of a pressure or a temperature resulting from the chemical substance or the hot water.

19. The sensing system according to claim 11, wherein each sensing device further comprises a coupler coupled to the device main body, for enabling the sensing device to work at the predetermined depth in the respective monitoring well, and the coupler has a quick connector to facilitate a connection between the device main body and the coupler.

20. A sensing method for use in civil engineering, comprising:
selecting a land to be measured;
determining a reference point on the land to be measured;
arranging a plurality of monitoring wells around the reference point according to a predetermined arrangement;
selecting a measurement point at a respective depth for each of the plurality of monitoring wells according to a predetermined plan;
respectively placing a plurality of sensing devices according to claim 1 at the measurement points; and
sensing an environmental parameter using the plurality of sensing devices.
